(19) United States
(12) Patent Application Publication
(10) Pub. No.: US 2007/0065518 A1
(43) Pub. Date: Mar. 22, 2007

(54) Colostrum compositions and methods

(76) Inventors: Harry Leneau, Jasper, MO (US); Judith Lenkau, Jasper, MO (US); Steven Allday, Shelbyville, KY (US)

Correspondence Address: ICE MILLER LLP, ONE AMERICAN SQUARE, SUITE 3100, INDIANAPOLIS, IN, 46282-0200, US

(21) Appl. No.: 10/576,841

(22) PCT Filed: Oct. 20, 2004
(86) PCT No.: PCT/US04/34770

§ 371 Date: Apr. 20, 2006

Related U.S. Application Data
(60) Provisional application No. 60/513,361, filed on Oct. 22, 2003.

(51) Int. Cl. A61K 35/20

(52) U.S. Cl. 424/535

(57) ABSTRACT

Colostrum products and a methods of using the colostrum products are provided. Methods are provided for preparing the colostrum products, which may be filtered and sterilized. The colostrum products may be injected, illustratively subcutaneously and intravenously, to provide an animal protection from disease.

### FIELD OF THE INVENTION

The present invention relates to a composition and method for providing protection against pathogens. More particularly, this invention provides compositions comprising colostrum for use in providing protection against pathogens.

### BACKGROUND AND SUMMARY OF THE INVENTION

Bovine Respiratory Disease Complex (BRD) is a multivalent disease of cattle, one segment of which is known as “shipping disease.” BRD is caused by both viral and bacterial pathogens, and more than 20 different viruses and approximately six common bacterial pathogens are associated with the disease. Typically, the bacterial challenges follow a viral challenge. Illustrative viral pathogens include Infectious Bovine Rhinotracheitis (IBR), Bovine Viral Diarrhea (BVD), Parainfluenza 3 (PI-3), and Bovine Respiratory Syncitial Virus (BRSV).

Colostrum is a substance secreted in the first few days post-partum prior to onset of true lactation. Colostrum contains proteins, carbohydrates, fats, vitamins, and minerals. In addition, colostrum contains bioactive components such as growth factors and antimicrobial factors. The antimicrobial factors include immunoglobulins, lactoperoxidase, lysozyme, and lactoferrin. Bovine colostrum is extremely rich in immunoglobulins. The concentration of IgG1 (52-87 g/l), IgG2 (1.6-2.1 g/l), IBM (3.7-6.1 g/l), and Riga 3.2-6.2 g/l) in bovine colostrum is approximately 100 fold higher than in normal bovine milk. Colostrum is routinely provided to calves, both for its nutritional and its antimicrobial effects. However, colostrum, by its nature, is not a sterile product, and its use has been generally limited to oral ingestion.

The present invention is directed to a colostrum product and a method of using the colostrum product. The colostrum product may be filtered and sterilized, and may be injected, illustratively subcutaneously and intravenously. Subcutaneous and intravenous injections of filtered sterile colostrum have been demonstrated to provide beneficial effects against Infectious Bovine Rhinotracheitis (IBR), Bovine Viral Diarrhea CBVD), Parainfluenza 3 (PI-3), and Bovine Respiratory Syncitial Virus (BRSV).

Additional features of the present invention will become apparent to those skilled in the art upon consideration of the following detailed description of the preferred embodiments.

### DETAILED DESCRIPTION OF THE INVENTION

In accordance with the present invention, a method is provided for providing an animal protection against pathogens. The method comprises delivering to the animal by injection a composition comprising an effective amount of a colostrum product. An “effective amount” as used herein refers to the amount of colostrum product which, upon injection, provides protection against pathogens. The colostrum product is illustratively colostrum that has been sterilized to provide a product that meets acceptable sterility requirements for injection. The colostrum used to make the colostrum product is also illustratively filtered to remove large components to provide a composition that is more compatible with injection. The animal illustratively may be a warm-blooded vertebrate, illustrative a bovine, ovine, equine, or porcine species, and the pathogens may be pathogens frequently encountered in commercial farming, breeding, or raising of the animal species. In one illustrative embodiment, the animal is a bovine calf, and the method is used to provide protection against IBR, BVD, PI-3, or BRSV. Illustratively, the colostrum is obtained from a post-partum female of the same species. However, it is understood that the colostrum product may be obtained from an animal of one species and used to provide protection to an animal of another species. Furthermore, it is understood that the animals discussed herein are illustrative only, and the colostrum product may be used to provide protection to other animals, particularly other warm-blooded vertebrates. Illustratively, the colostrum product may be used independently, or may be used in conjunction with a vaccination protocol.

### EXAMPLES

### Example 1

### Preparation of Sterile Highly Filtered Bovine Colostrum

Colostrum was obtained from Grade A dairy herds. The raw colostrum is filtered through a series of filters, illustratively starting with a 10 micron filter, followed by a 5 micron filter, and finishing with a 3 micron filter. Illustratively Millipore® filters (Billerica, Mass.) with polyester felt filter bags are used. The filtration removes large components, such as aggregates of lipids, proteins, and other materials, which may interfere with absorption or may result in sterile abscesses. Other filtration protocols, as are known in the art, may be used to remove the large components.

The filtered colostrum is packaged in containers and frozen. Sterilization is accomplished by 1.0 to 4.5 Mrad gamma-irradiation. Illustratively, the sterilization takes place on frozen or highly refrigerated colostrum, to prevent or minimize denaturation. While gamma-irradiation is used for sterilization of the illustrated embodiment, other methods of sterilization are contemplated and are within the scope of this invention. Such other methods include, but are not limited to, UV light and heat. Such methods may be time and/or temperature sensitive. Illustratively, the sterile product would be provided refrigerated.

Immunoglobulin levels in the sterile highly filtered colostrum were obtained from an independent lab (VMRD, Inc., Pullman, Wash.). IgG, IgA, and IgM levels do not vary significantly from those of the raw colostrum, as follows:

| | Raw Colostrum | Sterile Filtered Colostrum |
|---|---|---|
| | (mg/100 ml) | (mg/100 ml) |
| IgA | 250 | 240 |
| IgG | 4200 | 3700 |
| IgM | 190 | 170 |

These immunoglobulin levels are much higher than the serum immunoglobulin levels prior to treatment of the calves of the test group discussed below.

### Example 2

### Preparation of Composition

The sterile highly filtered colostrum was packaged without a carrier. However, standard carriers and exipients, as are known in the art may be used.

Dosages of 1 μl to 1000 ml may be provided, preferably, about 0.1 to 100 ml, most preferably about 25 to 75 ml. Dosages may be adjusted due to the size and species of the animal. In calves, a dose for a newborn animal may be 20-40 ml; in a 200-400 lb animal a dose of 40-60 ml may be used, and in larger calves of >400 lbs, a single dose of 100 ml may be provided, or several doses of 100 ml may be provided in multiple sites. Illustratively a dose of 50 ml is used.

### Example 3

### IBR Viral Challenge Subsequent to Subcutaneous Injection

Ten calves were used in this study. The calves were observed for ten days prior to commencement of the study, to insure that each calf is healthy.

Day 1: blood samples for viral titers were obtained, nasal swabs were obtained, and each calf was ear tagged. The calves were divided into two groups of five calves each. Each of the five calves in the test group were given a subcutaneous injection of 50 cc of the colostrum as prepared in Example 1. Each of the five calves in the control group were given a subcutaneous injection of 50 cc of fetal bovine serum, which was free of immunoglobulins.

Day 2: all ten calves were challenged with a live viral mixture containing IBR. 3.0 cc of the live virus was introduced into each nostril.

Days 3-8: nasal swabs were obtained from both sides of the nasal cavities of each calf. Each day the viral swabs were placed in viral transport medium, kept cold, and shipped overnight to the laboratory.

IBR virus shedding was reduced by 72% in the test group animals, as compared to the control animals. No test animals required any antibiotic treatment for symptoms. Among the control animals, one calf required no antibiotic treatment, three calves required two days of treatment, and one calf required four days of treatment.

The results of this study demonstrated a significant reduction in IBR virus shedding, as well as a significant reduction in symptoms.

### Example 4

### BVD Viral Challenge Subsequent to Subcutaneous Injection

This study was performed in the same manner as the study of Example 3, except that 3.0 cc of BVD was introduced into each nostril.

BVD virus shedding was reduced by 63% in the test group animals, as compared to the control animals. No test animals required any antibiotic treatment for symptoms. Among the control animals, one calf required two days of treatment, four calves required two days of treatment, and one calf required four days of treatment.

The results of this study demonstrated a significant reduction in BVD virus shedding, as well as a significant reduction in symptoms.

### Example 5

### PI-3 Viral Challenge Subsequent to Subcutaneous Injection

This study was performed in the same manner as the study of Example 3, except that 3.0 cc of PI-3 was introduced into each nostril.

PI-3 virus shedding was reduced by 81% in the test group animals, as compared to the control animals. One test animal required two days of antibiotic treatment. None of the other four test animals required any antibiotic treatment for symptoms. Among the control animals, all five calves required two days of treatment.

The results of this study demonstrated a significant reduction in PI-3 virus shedding, as well as a significant reduction in symptoms.

### Example 6

### BRSV Viral Challenge Subsequent to Subcutaneous Injection

This study was performed in the same manner as the study of Example 3, except that 3.0 cc of BRSV was introduced into each nostril.

BRSV virus shedding was reduced by 11% in the test group animals, as compared to the control animals. No test animals had any symptoms and none required any antibiotic treatment. Among the control animals, two calves had no symptoms and required no antibiotic treatment, two control group calves had elevated temperatures of 102-104° F. and loss of appetite for two days, and one control group calf had elevated temperatures of 103-104° F. and loss of appetite for two days.

The results of this study demonstrated a significant reduction in BRSV virus shedding, as well as a significant reduction in symptoms.

Although the invention has been described in detail with reference to certain preferred embodiments, those skilled in the art will recognize that the invention can be practiced with variations and modifications within the scope and spirit of the invention as described and defined in the following claims.

1. A composition comprising sterile bovine colostrum that has been highly filtered.

2. The composition of claim 1 provided in injectable form.

3. The composition of claim 2 provided in a syringe.

4. The composition of claim 2 further comprising a carrier suitable for injection.

5. A method of preparing sterile highly filtered colostrum comprising the steps of
filtering colostrum to remove large components while retaining antibodies, and
sterilizing the colostrum.

6. The method of claim 5 wherein the sterilizing step is performed by gamma irradiating the sample.

7. The method of claim 5 wherein the filtering step is performed by filtering the colostrum through a series of filters.

8. The method of claim 7 wherein the series of filters comprises a 10 micron filter, a 5 micron filter, and a 3 micron filter.

9. A method for providing an animal protection from disease comprising the step of
injecting animal with a dose of colostrum.

10. The method of claim 9 wherein the colostrum is sterilized.

11. The method of claim 9 or claim 10 wherein the colostrum is highly filtered.

12. The method of claim 9 wherein the animal is a warm-blooded mammal.

13. The method of claim 12 wherein the warm-blooded mammal is a bovine.

14. The method of claim 13 wherein the bovine is a calf.

15. The method of claim 12 wherein the warm-blooded mammal is a juvenile.

16. The method of claim 12 wherein the colostrum is obtained from an animal of the same species as the warm-blooded mammal.

17. The method of claim 9 further comprising the step of injecting the animal with a second dose of colostrum at a subsequent date.
